(19) United States
(12) Patent Application Publication
(10) Pub. No.: US 2016/0092793 A1
(43) Pub. Date: Mar. 31, 2016

(54) PHARMACOVIGILANCE SYSTEMS AND METHODS UTILIZING CASCADING FILTERS AND MACHINE LEARNING MODELS TO CLASSIFY AND DISCERN PHARMACEUTICAL TRENDS FROM SOCIAL MEDIA POSTS

(71) Applicant: Thomson Reuters Global Resources, Baar (CH)

(72) Inventors: Andrew G. Garrow, Milton Keynes (GB); Jochen L. Leidner, London (GB); Vasileios Plachouras, London (GB); Timothy C.O. Nugent, London (GB)

(21) Appl. No.: 14/861,714

(22) Filed: Sep. 22, 2015

Related U.S. Application Data
(60) Provisional application No. 62/065,933, filed on Oct. 20, 2014.
(60) Provisional application No. 62/065,247, filed on Oct. 17, 2014.
(60) Provisional application No. 62/055,911, filed on Sep. 26, 2014.

(51) Int. Cl. G06N 99/00; G06F 17/30

(52) U.S. Cl. CPC G06N 99/005; G06F 17/30867; G06F 17/3053

(57) ABSTRACT

Systems and methods for utilizing filters to reduce an incoming stream of textual messages to a smaller subset of potentially relevant textual messages, and using trained machine learning models to analyze and classify the content of such textual messages. Analyzed messages that belong to a relevant class as determined by the machine learning model are stored in a database, giving users the ability to determine and analyze trends from the subset of messages, such as adverse side effects caused by pharmaceuticals or the efficacy of pharmaceuticals. Relationships between the side effects caused by different pharmaceuticals can be used to predict potential candidates for drug repositioning.

### CROSS-REFERENCE TO RELATED APPLICATIONS

This application claims the benefit under 35 U.S.C. §119(e) of U.S. Provisional Patent Application No. 62/055,911, filed Sep. 26, 2014, U.S. Provisional Patent Application No. 62/065,247, filed Oct. 17, 2014, and U.S. Provisional Patent Application No. 62/065,933, filed Oct. 20, 2014, which are all hereby incorporated by reference in their entirety.

### FIELD OF THE INVENTION

Various embodiments of the present invention generally relate to pharmacovigilance systems and methods for filtering and classifying textual messages. More particularly, embodiments of the present invention generally relate to using cascading filters and machine learning models to filter and classify social media posts related to adverse reactions and side effects from pharmaceutical products and discussed in the social media posts. Embodiments of the present invention also relate to the use of global statistical models to predict candidates for drug repositioning from social media posts related to adverse reactions and side effects resulting from pharmaceuticals.

### BACKGROUND OF THE INVENTION

Conventional methods for detecting relationships between adverse side effects and particular pharmaceuticals initially rely on expensive, time-consuming clinical trials. However, the limited number of participants in these trials, as well as their time constraints, do not necessarily ensure that all adverse side effects of a particular drug will be identified. Once clinical trials conclude and a drug is released on the commercial market, pharmaceutical companies and medical professionals report adverse reactions to drugs to government authorities, and some countries operate systems that allow patients to directly report drug related adverse effects. This approach, however, results in under-reporting of drug related adverse side effects.

Each year, social media platforms such as FACEBOOK®, TWITTER®, and TENCENTWEIBO® (to name a few examples), grow increasingly popular, and the volume of information generated each day by users posting on these social media platforms has grown exponentially. For example, in 2014, users of TWITTER® alone generated approximately 500 million tweets every day at a rate of approximately 21 million tweets per hour. And the volume of such textual posts is expected to continue to grow, as more users join currently known or future social media platforms. Filtering through the amount of data generated on TWITTER® alone (not to mention other social media platforms) to identify messages that contain relevant information with regard to any particular topic or issue is a task that is inefficient and cost prohibitive to perform by human analysis.

We have discovered that automating the process of filtering and classifying social media data can advantageously be used to discern and analyze pharmacological trends and relationships. For example, we have discovered that automating the process of filtering and classifying social media data in connection with drug related adverse side effects (and other information of interest) associated with taking a particular pharmaceutical can advantageously be used to identify previously unknown relationships between drugs and side effects, and to monitor trends in those relationships, for example, chronologically and/or geographically. We have also discovered that early identification of such drug related adverse side effects will improve the well-being of patients, and reduce the costs incurred by health systems and patients to treat such side effects. We have further discovered that collecting and classifying social media posts that discuss drug related side effects can be used to predict new therapeutic applications for existing drugs, a process known as “drug repositioning.”

In addition, we have discovered that automating the process of filtering and classifying social media data can be used to identify trends and relationships in the efficacy of pharmaceuticals (e.g., the ability of a medical drug to produce a desired or intended treatment result), professional and patient feedback on drugs, and other drug related information. And we have also discovered that automating the process of filtering and classifying social media data can be used to identify trends and relationships in connection with medical devices or surgical procedures.

### SUMMARY OF EMBODIMENTS OF THE INVENTION

To address these and/or other needs, systems and methods are provided to discern and analyze pharmacological trends and relationships. One exemplary system includes a server operatively configured to receive a plurality of textual messages. The server includes a plurality of cascading filters, wherein the plurality of textual messages are input into a first cascading filter, and each of the cascading filters evaluates whether textual messages input into that filter satisfy a criterion of that filter. Each of the plurality of cascading filters outputs a subset of textual messages that satisfy the criterion of that filter, so that a last cascading filter outputs a final subset of the plurality of textual messages. The server also includes a feature extractor that receives the final subset of textual messages, extracts a vector of features from each textual message of the final subset, and outputs the final subset of textual messages and an associated vector of features for each message of the final subset.

The server also utilizes a classifier that includes a machine learning model that receives the vectors of features, and determines whether the textual message associated with each vector of features belongs to a particular class associated with the machine learning model. The classifier provides an output of one or more textual messages that belong to that particular class to an indexed database of classified textual messages that stores the classified textual messages, a particular class associated with those classified textual messages, and metadata associated with those classified textual messages.

The content of the indexed database can be utilized in various ways and can be provided in one or more data formats to a client application through an application programming interface (API). In one embodiment information and/or data of the indexed database can be displayed in one or more visual representations in response to a search request to the system. In another embodiment the data can be visualized based on a frequency of side effects of one or more medical drugs over time. In yet another embodiment the data can also be visualized as an association strength between one or more side effects of one or more medical drugs.

The indexed database can be searched based on a medical or pharmaceutical drug name, a side effect name, a time interval, a geographic region, and/or a geographic location. One or more results in response to a search can be displayed or further processed.

The exemplary system can also be used to predict candidates for drug repositioning by collecting textual messages discussing drug-related side effects, generating side effect profiles for a number of drugs discussed in those textual messages, and calculating correlations between the side-effect profiles of these drugs to predict which drugs might share a common mechanism of action.

While exemplary embodiments pertain to classifying messages relating to drugs and pharmaceuticals and predicting candidates for drug repositioning, it will be recognized that the disclosed systems and methods can be generally used in connection with filtering and classifying textual messages dealing with any subject area of interest. For example, the disclosed systems and methods can also be used in connection with recognizing textual messages relevant to medical devices, diseases, diagnostics, therapies, or other non-medical areas of interest.

### BRIEF DESCRIPTION OF THE DRAWINGS

FIG. 1 is a diagram of an exemplary system for filtering and classifying textual messages.

FIG. 2 is a flow diagram of an exemplary method performed by the system of FIG. 1.

FIG. 3 is a diagram of an exemplary embodiment of one of the feature vectors depicted in FIG. 1.

FIG. 4A is a diagram of an exemplary cascaded embodiment of the classifier depicted in FIG. 1.

FIG. 4B is a diagram of an exemplary parallel-voting embodiment of the classifier depicted in FIG. 1.

FIG. 5 is a flow diagram depicting an exemplary procedure for training the machine learning model of the classifier depicted in FIG. 1.

FIG. 6 is a depiction of an exemplary graphical user interface generated by the customer application depicted in FIG. 1.

FIG. 7 is a flow diagram depicting an exemplary method for filtering and classifying textual messages in order to predict potential candidates for drug repositioning.

FIG. 8 is a diagram of an exemplary system for predicting candidates for drug repositioning using social media posts discussing drug-related side effects.

FIG. 9 is a flow diagram depicting an exemplary method performed by the system of FIG. 8.

FIG. 10 is a depiction of an exemplary graphical display generated by the graphical model generator of the system of FIG. 8.

### DETAILED DESCRIPTION OF EXEMPLARY EMBODIMENT(S)

The figures and descriptions have been provided to illustrate elements of the present invention, while eliminating, for purposes of clarity, other elements found in a typical communications system that may be desirable or required to facilitate use of certain embodiments. For example, the details of a communications infrastructure, such as the Internet, a cellular network, and/or the public-switched telephone network are not disclosed. However, because such elements are well known in the art, and because they do not facilitate a better understanding of the present invention, a discussion of such conventional elements is not included.

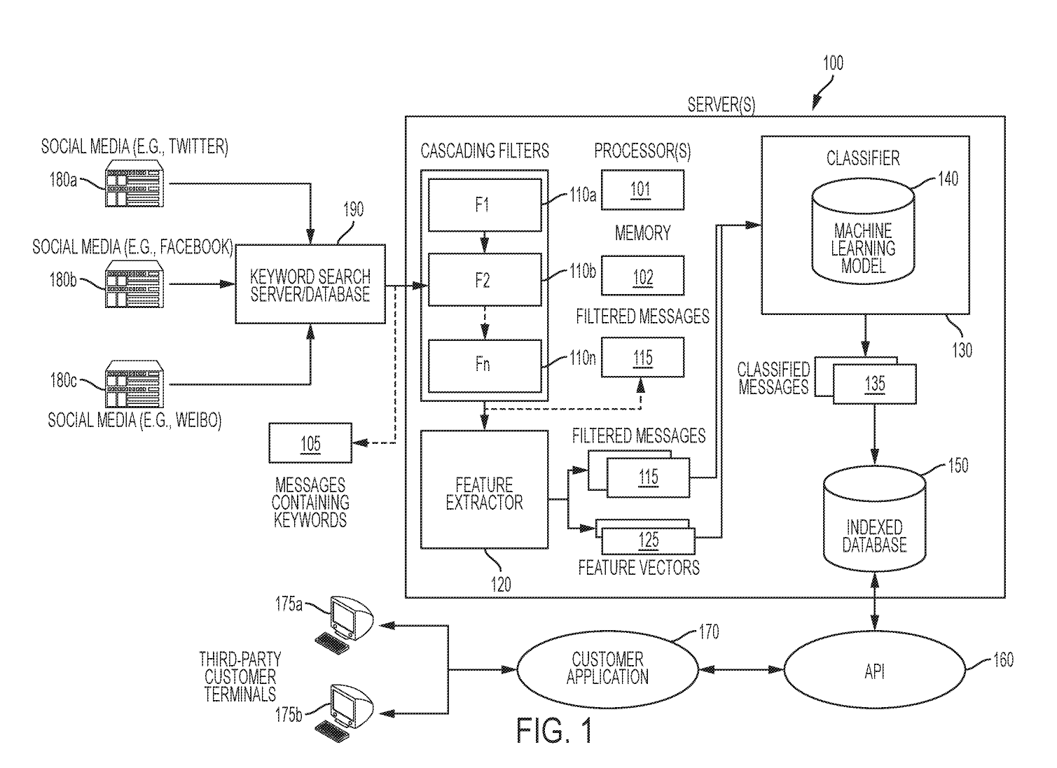

FIG. 1 is a diagram of an exemplary system for filtering and classifying textual messages. Server 100 includes at least one memory unit 102 and at least one processor 101, and hosts the plurality of cascading filters 110, a feature extractor 120, classifier 130, and indexed database 150. In some embodiments, server 100 may be a single server 100 featuring one or more processors 101 and one or more memories 102.

Server 100 may also consist of a plurality of servers. For example, cascading filters 110, feature extractor 120, classifier 130, and indexed database 150 may each be hosted on a separate server. In addition, one or more of cascading filters 110, feature extractor 120, classifier 130, and indexed database 150 may be distributed over two or more separate servers. For example, filters 110a, 110b, and 110n may each be hosted on one or more separate servers, and/or indexed database 150 may be hosted on two or more separate servers.

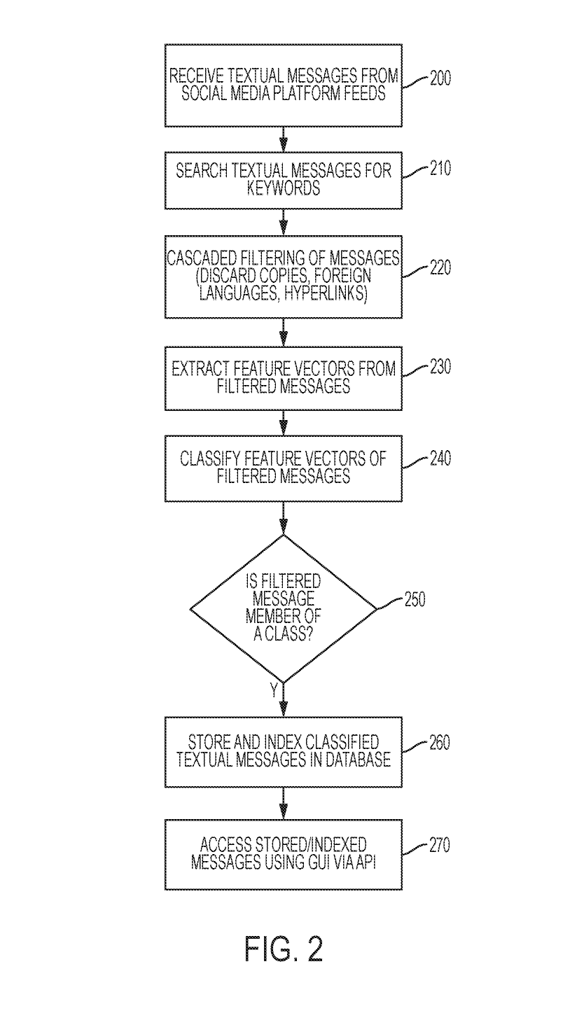

In FIG. 1, social media platforms 180a, 180b, and 180c provide a stream of posts by users to keyword search server 190, as depicted in step 200 in FIG. 2. The social media posts provided by social media platforms 180a, 180b, and 180c are textual messages written by the users of social media platforms 180a, 180b, and 180c. Each of social media platforms 180a, 180b, or 180c may provide keyword search server 190 with all of the posts from that social media platform 180a, 180b, or 180c (e.g., in the case of TWITTER®, the so-called “full firehose” feed of data) or a subset of the posts from that social media platform 180a, 180b, or 180c (e.g., in the case of TWITTER®, the subset of tweets provided by the TWITTER® API).

Keyword search server 190 can be operated by the same entity that operates server 100, or by a third party vendor who provides server 100 with social media posts 105 that contain one or more keywords. Keyword searching can also be performed by server 100.

Keyword search server 190 may be a single server or a number of servers that receive and search posts from social media platforms 180a, 180b, and/or 180c. Keyword search server 190 may include or utilize one or more databases to store social media posts 105 that contain one or more keywords of interest.

In some embodiments, keyword search server 190 may contain a list of keywords that the server 190 uses to search the textual messages provided by social media platforms 180a, 180b, and 180c, as depicted by step 210 of FIG. 2. This list of keywords may also be contained in a database hosted on keyword search server 190.

In an embodiment of keyword search server 190 that receives social media posts that contain descriptions of adverse side effects associated with a drug, the list of keywords utilized by keyword search server 190 may include, for example, a list of drug brand names, the generic names for or active ingredients of those brand name drugs, and/or a list of phrases indicating side effects associated with those drugs (e.g., “anxiety attack,” “appetite,” “bleed,” “bone pain,” “constipation,” “cotton mouth,” “dizzy,” “drooling,” “drowsy”, “dry mouth,” “faint,” “fatigue,” “gain weight,” “hallucination,” “heart disease,” “hives,” “hypertension,” “itchy,” “joint pain,” “malaise,” “memory loss,” “mood swing,” “nausea,” “nightmare,” “palpitation,” “panic attack,” “vomit,” “and “weakness”). The keyword search can reduce the number of social media posts (in this case, TWITTER® posts) from approximately 500 million messages per day to approximately 179,000 messages per day. This keyword search, therefore, only selects a subset of approximately 0.0358% of the total TWITTER® posts each day for further review. The number of social media posts searched may increase or decrease depending, for example, on the number of social media networks searched, the number of users of those social media networks, the volume of posts generated by those users, and network capacity and/or bandwidth. Similarly, the percentage of messages identified by keyword search server 190 may vary depending, for example, on the number of keywords used to search and the popularity of those keywords.

Instead of a list of defined keywords, keyword search server 190 may collect all social media posts containing a word or phrase that matches at least one morphological structure. For example, in the embodiment of keyword search server 190 that receives social media posts that contain descriptions of adverse side effects associated with a drug, keyword search server 190 may collect all textual messages containing a word or phrase that matches the American Medical Association's prefix, infix, and stem morphological structure for the naming of generic drugs.

After searching the posts provided by social media platforms 180a, 180b, and 180c, keyword search server 190 provides social media messages 105 containing keywords of interest to server 100 for further filtering and analysis. Server 100 receives keyword-containing messages 105, and inputs those messages 105 into a system of cascading filters 110 to further filter out irrelevant messages, as depicted by step 220 of FIG. 2.

Cascading filters 110 can contain a number of separate filters. While FIG. 1 depicts three filters 110a, 110b, and 110n, the set of cascading filters 110 may contain more or fewer than the depicted three filters depending on the set of criteria for producing a set of filtered messages 115. For example, in some embodiments, instead of separate keyword search server 190, server 100 may have a keyword search filter in the set of cascading filters 110 that filters out all textual messages that do not contain a keyword or phrase of interest.

Each filter 110a, 110b, or 110n has a unique criterion. If a message 105 input into filter 110a meets the criterion of filter 110a, it is passed through to the next filter 110b. If the message 105 does not meet the criterion of filter 110a, it is discarded. Next, if message 105 has been passed through to filter 110b and meets the criterion of filter 110b, it is passed through to final filter 110n. If it does not meet the criterion of filter 110b, it is discarded. If message 105 has been passed through to final filter 110n of cascading filters 110, and meets the criterion of filter 110n, it is output from the set of cascading filters 110 as a filtered message 115 and provided to feature extractor 120. If message 105 does not meet the criterion of filter 110n, it is discarded.

In some embodiments, one of filters 110a, 110b, and 110n is a filter that outputs only original social media posts, discarding all social media posts that are copies of those original posts. For example, if the social media posts 105 input into filter 110a, 110b, and 110n are TWITTER® posts, the filter 110a, 110b, and 110n will output original tweets while discarding all retweets. In an embodiment of a system designed to collect social media posts from TWITTER® 105 about adverse side effects of a drug, only the original tweets about adverse side effects would be of interest, not the retweets of those original tweets (which would be false positives).

In some embodiments, one of filters 110a, 110b, and 110n is a filter that outputs only social media posts that do not contain hyperlinks, discarding all social media posts that contain hyperlinks. In these embodiments, it has been observed that social media posts that contain hyperlinks have a higher likelihood of being commercial spam or non-informative textual messages in comparison to social media posts that do not contain hyperlinks.

In some embodiments, one of filters 110a, 110b, and 110n is a filter that outputs only messages written in a single particular language, while discarding messages not in that language. Because a machine learning model 140 of classifier 130 is optimized for textual messages in a particular language, if classifier 130 contains only one or more machine learning models 140 that are optimized for a single particular language, classifier 130 will not be able to classify textual messages that are not in that language, allowing them to be discarded by the set of cascading filters 110. If classifier 130 contains machine learning models 140 that are each capable of classifying textual messages in a different language, however, the set of cascading filters 110 should output filtered textual messages 115 that are composed in any of those different languages (while still discarding messages that are composed in a language other than those different languages).

In embodiments where the set of cascading filters 110 contains a filter 110a, 110b, or 110n that outputs only messages in one (or more) specific language(s), the filter 110a, 110b, or 110n may utilize the off-the-shelf language identification tool “langid.py.”

In an embodiment, the set of cascading filters 110 can receive approximately 179,000 TWITTER® posts 105 per day containing matching keywords, and is made up of an initial filter 110a which filters out all messages 105 which are copies of original messages, a second filter 110b which filters out all messages 105 containing hyperlinks, and a third filter 110n which filters out all messages 105 which are not written in English. In this embodiment, the set of cascading filters 110 reduces the amount of TWITTER® posts from an average of approximately 179,000 messages 105 per day to approximately 26,000 filtered messages 115 per day. In this embodiment, the set of cascading filters 110 filters out approximately 85.5% of messages containing keywords 105. The set of cascading filters 110 may be used to filter any number of messages containing keywords 105, however, and the percentage of messages 105 that are filtered out may vary depending on the number of cascading filters 110 and the extent to which messages 105 meet the criteria of those filters 110.

Filtered messages 115 are provided as an input to and received by feature extractor 120. For each filtered message 115, feature extractor 120 extracts a pattern describing the content of that filtered message 115, as depicted by step 230 of FIG. 2. In embodiments of the invention, the pattern xε [symbol] ^{d} describing the content of filtered message 115 is a d-dimensional vector of features 125 extracted for that message 115 by feature extractor 120. By comparing the pattern 125 for a message 115 against the patterns of previous messages contained in machine learning model 140, classifier 130 can determine whether filtered message 115 having feature vector 125 is a member of a particular class, as depicted by steps 240 and 250 of FIG. 2, by associating feature vector 125 with a response value yε{−1, +1}, where +1 indicates that the message 115 corresponding to the vector 125 is a member of a class and −1 indicates that the message 115 is not a member of that class.

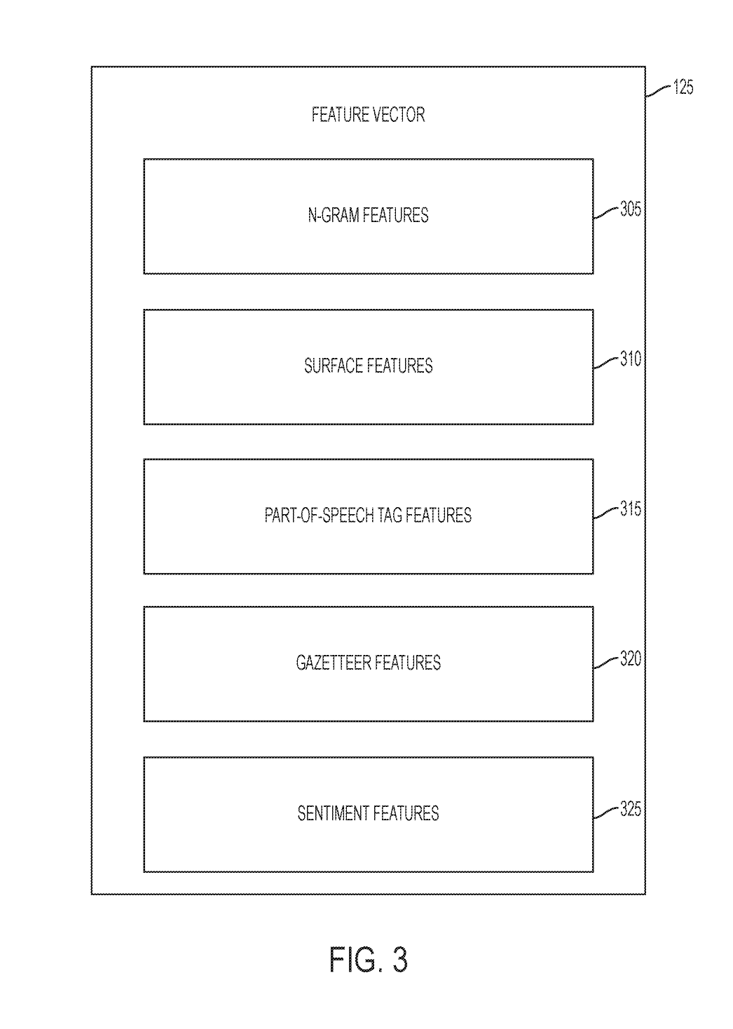

In an embodiment, feature extractor 120 analyzes filtered social media posts 115 from TWITTER®, and extracts a feature vector 125 from each tweet 115 output by the set of cascading filters 110. This feature vector 125, as shown in FIG. 3, contains N-gram features 305, surface features 310, part-of-speech tag features 315, gazetteer features 320, and sentiment features 325.

To extract N-gram features 305, feature extractor 120 tokenizes the text of tweet 115, and normalizes the text of tweet 115 by lowercasing each token in tweet 115. Next, feature extractor 120 extracts all unigrams and bigrams from the text of tweet 115, and keeps the ones that contain alpha-numeric characters. Feature extractor 120 generates binary indicator features BIN_NGRAML_w, which are set equal to 1 if tweet 115 contains an n-gram w with length L, and set equal to 0 otherwise. For example, for the text “I took two pills” for w=ε{1,2}, feature extractor 120 would generate the set of unigrams {i, took, two, pills} and the set of bigrams {i_took, took_two, two_pills}.

Feature extractor 120 also extracts surface features 310 from tweet 115, which can prove useful in extracting elements from the context of a user, such as their emotional state, engagement in discussions with other users, or their attitude towards an issue they had experienced.

In one embodiment, feature extractor 120 extracts the following exemplary text surface features from tweet 115: a) the number of characters in tweet 115 divided by the maximum length in characters of tweet 115 (e.g., 140 characters). Longer tweets 115 are more likely to be informative; b) the number of mentions (e.g., @Username) found in tweet 115. The presence of user mentions in tweet 115 indicates that there is a conversation between users; c) the maximum number of times a character is repeated within a token. This feature will have a high value when a user emphasizes a word by repeating a character several times, for example writing “sleeeepy” instead of “sleepy;” d) a binary feature set equal to 1 if tweet 115 contains at least one numerical token, such as in the phrase “I took 2 aspirin tonight;” e) a binary feature which is set equal to 1 if tweet 115 contains at least one title-case token, for example the word “TWITTER®;” and f) a binary feature which is set equal to 1 if tweet 115 contains at least one token with mixed capitalization, like “InterCity.”

Feature extractor 120 also extracts features 315 based on part-of-speech (POS) tags assigned to tokens in order to encode information related to the grammatical structure of tweet 115, for example, whether the writer of tweet 115 was asking a question or making a comparison. A POS tagger in feature extractor 120 adds POS tags to each token of tweet 115. The following table lists the types of POS tags and their description:

| Tag | Description |
|---|---|
| CC | Coordinating conjunction |
| CD | Cardinal number |
| DT | Determiner |
| EX | Existential there |
| FW | Foreign word |
| IN | Preposition or subordinating conjunction |
| JJ | Adjective |
| JJR | Adjective, comparative |
| JJS | Adjective, superlative |
| LS | List item marker |
| MD | Modal |
| NN | Noun, singular or mass |
| NNS | Noun, plural |
| NNP | Proper noun, singular |
| NNPS | Proper noun, plural |
| PDT | Predeterminer |
| POS | Possessive ending |
| PRP | Personal pronoun |
| PRP$ | Possessive pronoun |
| RB | Adverb |
| RBR | Adverb, comparative |
| RBS | Adverb, superlative |
| RP | Particle |
| SYM | Symbol |
| TO | to |
| UH | Interjection |
| VB | Verb, base form |
| VBD | Verb, past tense |
| VBG | Verb, gerund or present participle |
| VBN | Verb, past participle |
| VBP | Verb, non-3rd person singular present |
| VBZ | Verb, 3rd person singular present |
| WDT | Wh-determiner |
| WP | Wh-pronoun |
| WP$ | Possessive wh-pronoun |
| WRB | Wh-adverb |

In one embodiment, feature extractor 120 extracts the following exemplary text surface features from the tweet 115 based on the POS tags of the tokens of that tweet 115: a) a binary feature (past-present verbs) indicating whether tweet 115 contains verbs in both past and present tense. The feature value is set equal to 1 if tweet 115 contains verbs [symbol] ₁ and [symbol] ₂, where [symbol] ₁!= [symbol] ₂ and POS ( [symbol] ₁) in {VB, VBD, BDN} and POS ( [symbol] ₂) in {VB, VBP, VBG}, and otherwise 0; b) a binary indicator feature (question tags) which is set equal to 1 if tweet 115 contains word [symbol] for which POS ( [symbol] ) in {WDT, WP, WP#, WRB}, otherwise 0; c) a binary indicator feature (comparative-superlative tags) which is set equal to 1 if tweet 115 contains word [symbol] for which POS ( [symbol] ) in {BR, JJS}, otherwise 0; and d) a concatenation of all verb POS tags from tweet 115 in alphabetical order (verb signature).

Feature extractor 120 can also extract gazetteer features 320 from tweet 115. In an embodiment in which feature extractor 120 extracts features 320 relevant to whether the tweet 115 contains information about pharmaceuticals, feature extractor 120 utilizes three sets of gazetteers (lexicons), namely user vocabulary, company, and medical gazetteers.

The user vocabulary gazetteers are lists of words and phrases indicating abuse, humor, fiction, intake, efficacy, as well as patient feedback about a drug. The company gazetteers include lists of words related to commercial spam, commercial pharmaceutical companies, financial and share price information, company news, and company designators. The medical vocabulary includes gazetteers related to human body parts, adverse effect symptoms, side effect symptoms, adverse events, casuality indicators, clinical trials, medical professional roles, side effect triggers, and drugs.

In one embodiment, for each gazetteer, feature extractor 120 computes the following exemplary features 320: a) BIN_G: a binary feature set equal to 1 if tweet 115 contains at least one token matching an entry from gazetteer G; b) NUM_TOKENS_G: the number of tokens matching entries from gazetteer G; c) PRCNT_CHARS_G: the fraction of the number of characters in tokens matching entries from gazetteer G relative to the total number of characters in tweet 115.

On one embodiment, feature extractor 120 also extracts sentiment features 325 from tweet 115. The sentiment of users as expressed in their tweets 115 is potentially an important indication regarding the items mentioned in their tweet 115. To calculate user sentiment, in one embodiment, feature extractor 120 employs a dictionary which assigns each word in the dictionary a valence value between −5 and +5. To focus on words expressing strong sentiments, feature extractor 120 only takes into account dictionary entries having a valence greater than +2 or less than −2. During feature extraction, each word in tweet 115 is assigned a valence rating, and the positive and negative ratings are aggregated separately.

Feature extractor 120 can then generates the following exemplary features: a) F_OF_NEGATIVE_PHRASES: the number of tokens with a negative index, their sum, and their average; and b) F_OF_NEGATIVE_PHRASES: the number of tokens with a positive index, their sum, and their average.

For example, for a tweet 115 containing the word “better” and no negative words, feature extractor 120 would compute three sentiment features: the number of positive phrases (equal to 1), the sum of positive phrases (equal to the valence rating of “better,” +3), and the average of the positive phrases (also equal to the valence rating of “better,” +3).

Once feature extractor 120 has extracted feature vector 125 from message 115, the filtered message 115 and its associated feature vector 125 are provided to classifier 130. Classifier 130 is made up of one or more machine learning models 140, each of which has been trained to recognize feature vectors 125 that belong to a particular class of messages 135. In embodiments of the invention, machine learning model 140 is a support vector machine (SVM).

An SVM 140 is a non-probabilistic binary linear classifier. Each SVM 140 is trained to recognize messages 115 that are part of a particular class (for example, messages describing adverse side effects) and mark those messages 135 as positive examples of the class, while marking all other messages as negative examples (regardless of whether those messages are part of a different class). Therefore, a classifier 130 with a single SVM 140 is only capable of classifying a single class of messages 135, whereas a classifier 130 having multiple SVMs 140 is capable of classifying multiple classes of messages 135.

For example, classifier 130 having seven SVMs 140 could classify messages 115 that fall into any of the seven classes consisting of: 1) online vendors (does message 115 advertise for an online pharmacy/online business?); 2) patient feedback (does message 115 constitute feedback from a patient about the cost or availability of a pharmaceutical?); 3) professional feedback (does message 115 contain feedback from a doctor, scientist, pharmacist, or other medical professional?); 4) adverse event (does message 115 discuss a side effect of a pharmaceutical?); 5) efficacy (does message 115 discuss the effects or degree of effectiveness of a pharmaceutical?); 6) clinical trial (does message 115 discuss a clinical trial of a pharmaceutical?); and 7) pharma news (does message 115 constitute a piece of pharmaceutical news?).

In an embodiment, classifier 130 has a single SVM 140 trained to recognize TWITTER® messages 135 that contain discussion of the adverse effects of a drug. For example, classifier 130 analyzes approximately 26,000 filtered TWITTER® messages 115 per day (filtered from the approximately 179,000 TWITTER® messages 105 containing relevant keyword(s), those 179,000 messages 105 themselves collected from the approximately 500 million TWITTER® messages generated each day). From these 26,000 filtered TWITTER® messages 115 per day, the single SVM 140 of classifier 130 outputs approximately 82 positive examples of adverse event messages 135 per day to be stored in indexed database 150. In this embodiment, therefore, classifier 130 classifies approximately 0.3% of filtered messages 115 as positive examples of adverse event messages 135, and approximately only 0.0000164% of all the 500 million TWITTER® messages generated each day as positive examples of adverse event messages 135.

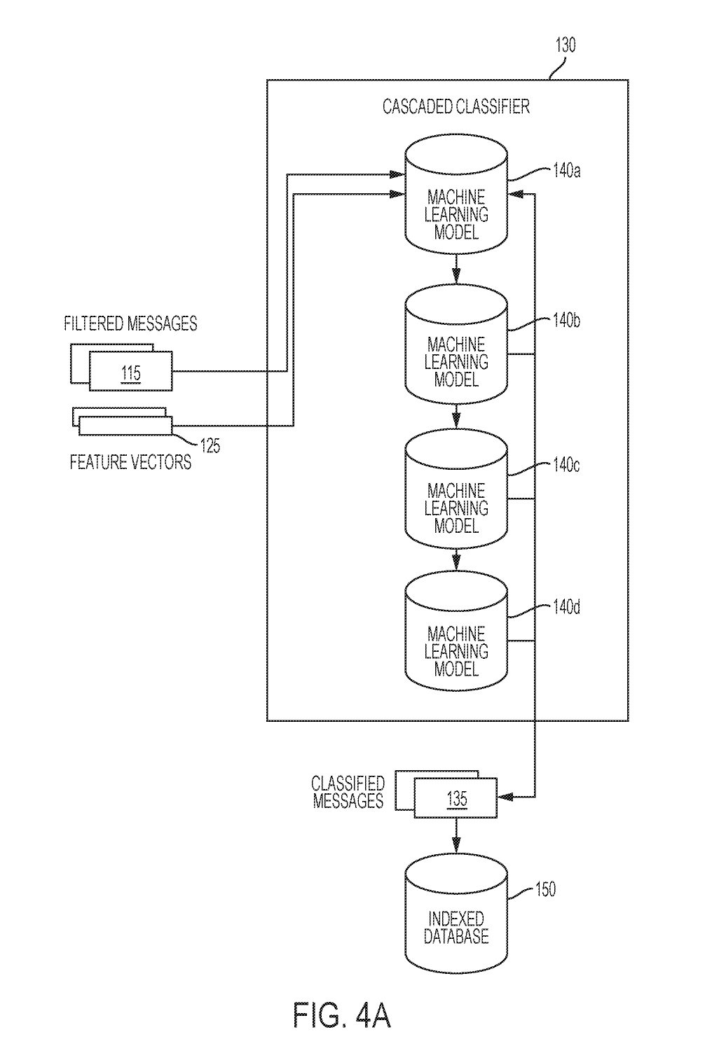

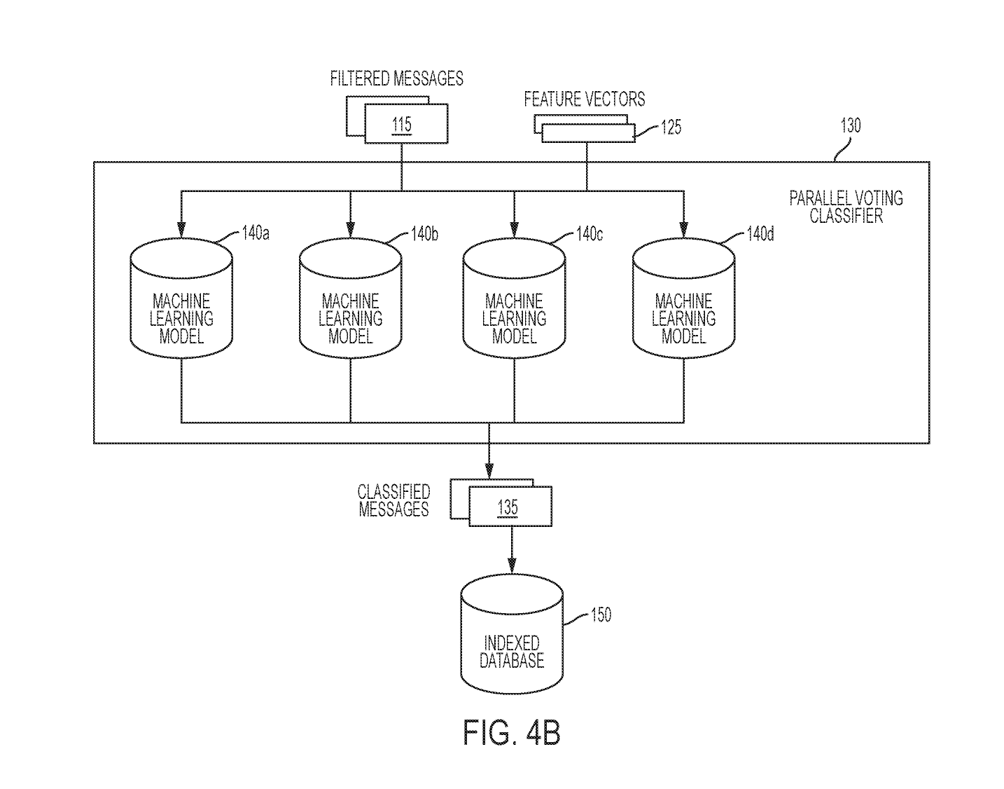

FIG. 4A is a diagram of an exemplary cascaded embodiment of the classifier depicted in FIG. 1, and illustrates an embodiment of classifier 130 having multiple SVM machine learning models 140. In this embodiment, classifier 130 is a cascaded classifier having machine learning models 140a, 140b, 140c, and 140d in series. The feature vectors 125 of filtered messages 115 are first input into first machine learning model 140a.

If SVM 140a determines that feature vector 125 corresponds to a first class of messages that SVM 140a has been trained to recognize, it outputs the classified message 135 to an indexed database of classified messages 150. If SVM 140a instead determines that feature vector 125 does not belong to the class of messages that SVM 140a has been trained to recognize, it instead classifies that feature vector 125 as a negative example, and passes the feature vector 125 on to SVM 140b. SVM 140b performs the same process for a second class of messages that SVM 140b has been trained to recognize, outputting positive examples 135 to database 150 and negative examples to SVM 140c, and SVMs 140c and 140d perform similar processes. If SVM 140d, the last machine learning model 140 in the cascaded classifier 130, classifies a feature vector 125 as a negative example, then that feature vector 125 and its associated filtered message 115 are discarded.

FIG. 4B is a diagram of an exemplary parallel-voting embodiment of the classifier depicted in FIG. 1, and illustrates another embodiment of classifier 130 having multiple SVM machine learning models 140. In this embodiment, classifier 130 is a parallel voting classifier featuring machine learning models 140a, 140b, 140c, and 140d in parallel. In parallel voting classifier 130, a feature vector 125 associated with a filtered message 115 is input into each of machine learning models 140a, 140b, 140c, and 140d in parallel.

In parallel voting classifier 130, if none of machine learning models 140a, 140b, 140c, and 140d classify feature vector 125 as a positive example, then that feature vector 125 and its associated filtered message 115 are discarded. If a single one of machine learning models 140a, 140b, 140c, and 140d classifies feature vector 125 as a positive example of the class that machine learning model 140a, 140b, 140c, or 140d has been trained to recognize, then the message 135 is classified as an example of that class and is output to indexed database 150.

If two or more of machine learning models 140a, 140b, 140c, and 140d each classify a single feature vector 125 as positive examples of the classes that those machine learning models 140a, 140b, 140c, and 140d have been trained to recognize, those two or more machine learning models 140a, 140b, 140c, and 140d vote on how confident each of the machine learning models 140a, 140b, 140c, or 140d is that the feature vector 125 is an example of the class that each respective model 140a, 140b, 140c, or 140d has been trained to recognize. The model 140a, 140b, 140c, or 140d with the highest confidence score “wins,” and the message 135 is classified as an example of the “winning” model 140a, 140b, 140c, or 140d's class and is output to indexed database 150.

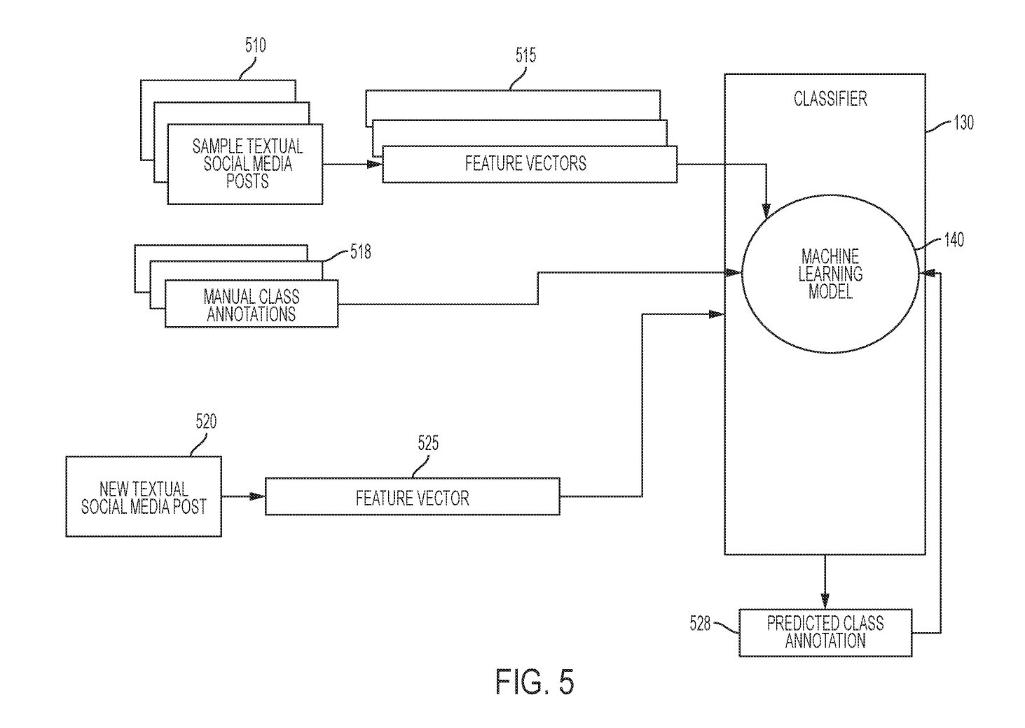

FIG. 5 is a flow diagram depicting an exemplary procedure for training the machine learning model of the classifier depicted in FIG. 1, and illustrates the training process for an SVM machine learning model 140. First, a number of feature vectors 515 are extracted (by feature extractor 120) from a number of sample textual messages 510, messages 510 which have been associated with manually created annotations 518 indicating whether messages 510 are positive or negative examples of the class that SVM machine learning model 140 is being trained to recognize.

The training process by which an SVM 140 learns to recognize messages that are members of a particular class is equivalent to the following optimization problem:

$${min}_{w ∈ {ℝ}^{d} , b ∈ ℝ , {ξ}_{i} ∈ ℝ +} ( {w}^{⊤} w + {C}^{+} {\sum }_{{y}_{i} = + 1} {ξ}_{i} + {C}^{-} {\sum }_{{y}_{i} = - 1} {ξ}_{i} )$$

subject to

yi(w^{T}φ(xᵢ)+b)≧1−ξᵢ and ξᵢ≧0 for i=1, . . . ,N.

The SVM 140 maps each of the sample feature vectors 515 as points in n-dimensional space. By associating a manual annotation 518 with each sample feature vector 515 to annotate that sample vector 515 is a positive or negative example of a class, the SVM 140 is able to define a dividing line in that n-dimensional space that divides positive example vectors 515 from negative example vectors 515. As new feature vectors 525 are extracted from unannotated messages 520 (by feature extractor 120), the SVM 140 can map the new feature vector 525 in the n-dimensional space, discern which side of the dividing line the feature vector 525 falls on, and create an annotation 528 for the textual message 520 as a positive or negative example of the class that the SVM 140 has been trained to recognize. The annotation 528 created by the SVM 140, if positive, can then itself be assessed by a human operator and manually corrected if the annotation 528 is a false positive, further training SVM 140 to omit such false positives in the future.

In addition to training the SVM 140 with manually annotated messages 510 (so-called “gold” training data), the SVM 140 may be trained using surrogate learning. Once the SVM 140 has been trained to an extent with the “gold” manually annotated messages 510, a set of “silver” data is generated, consisting of messages that have been automatically parsed and designated as likely positive examples of the class that the SVM 140 is being trained to recognize. This “silver” data can then be input into the SVM 140 to expand the set of training data for that SVM 140.

In addition to providing the SVM 140 with training data 510, the parameters of the SVM 140 may be tuned using grid search optimization to optimize the SVM 140's capability to accurately classify textual messages 520.

After classification, as discussed above, the classified textual messages 135 are indexed and stored in database 150, as depicted in step 260 of FIG. 2. In addition to the class of the messages 135 and the textual content of those messages 135, other metadata associated with messages 135 can be indexed and stored in database 150. Such metadata can include, for example, the time and date a message 135 was generated, the geographical location where a message 135 was generated, and/or demographical information about a user who generated a message 135, such as that user's age or gender.

An application programming interface 160 allows third-party users to access the indexed messages 135 and associated metadata stored in database 150 via one or more customer applications 170, as depicted by step 270 of FIG. 2. Alternatively, customer applications 170 may access the indexed messages 135 and associated metadata stored in database 150 directly without using application programming interface 160. Third-party users may run these customer applications 170 on terminals 175a and 175b, terminals 175a and 175b which may be any of a desktop computer, a laptop computer, a smartphone, a tablet, or other suitable computing devices.

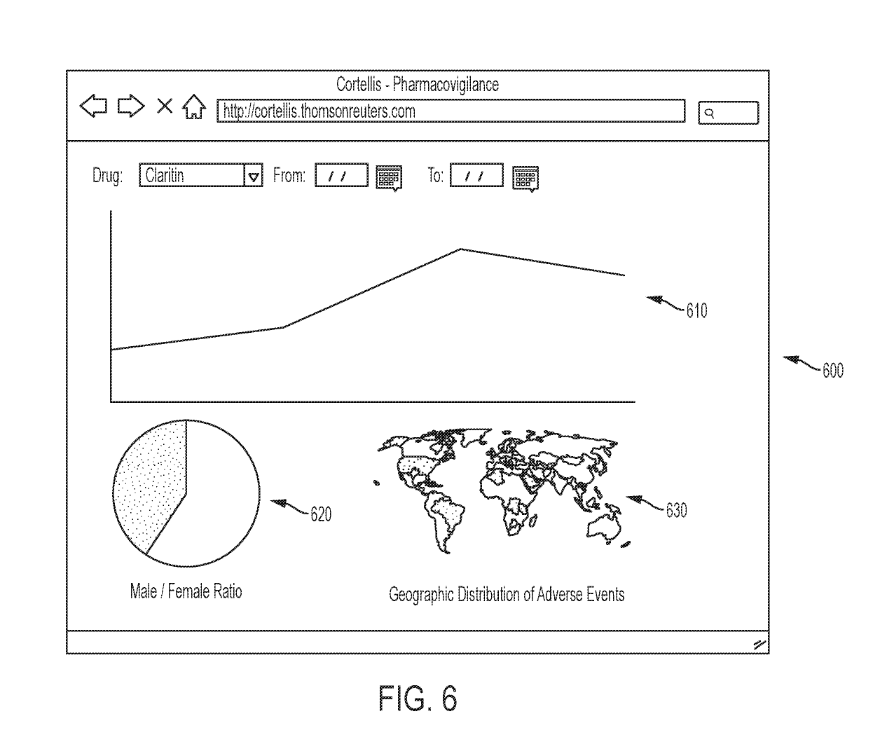

Customer application 170 may generate a graphical user interface configured to visually display the data stored in indexed database 150 on the displays of third-party user terminals 175a and 175b. FIG. 6 is a depiction of an exemplary graphical user interface generated by the customer application depicted in FIG. 1. FIG. 6 shows a chronological graph view 610 allowing a user to view the volume of classified messages 135 related to a particular pharmaceutical over time, a chart view 620 illustrating the gender makeup of users posting classified messages 135 related to that particular pharmaceutical, and a geographic view 630 illustrating the geographical distribution from where classified messages 135 related to the particular pharmaceutical were posted.

In embodiments, graphical user interface 600 will allow third-party users to view individual textual messages 135 that have been classified as part of a particular class. Users may be able to indicate using graphical user interface 600 whether they believe a particular message 135 was properly classified by the classifier 130, providing additional manual feedback for machine learning model 140 as depicted in FIG. 5.

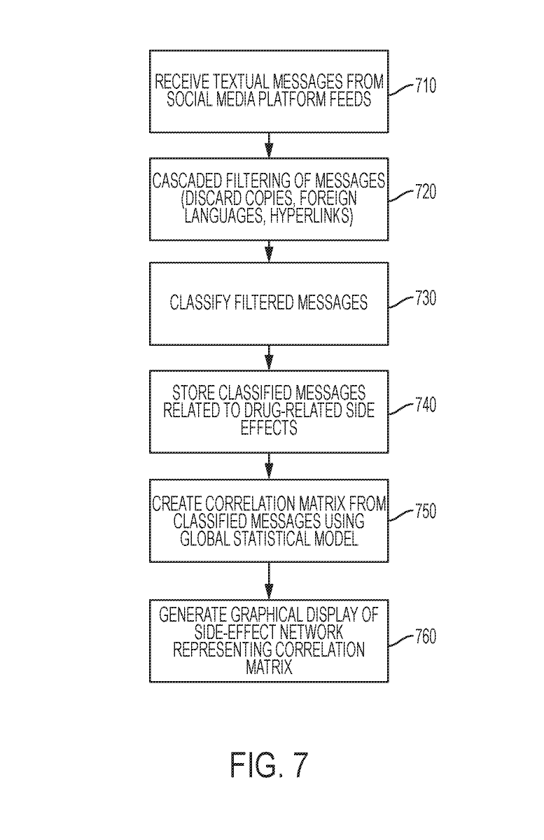

FIG. 7 is a flow diagram depicting an exemplary method for filtering and classifying textual messages in order to predict potential candidates for drug repositioning, and illustrates an exemplary use for the filtering and classification system described above: using social media to predict candidates for drug repositioning. Drug repositioning refers to the process of identifying novel therapeutic uses for already-marketed drugs that have existing therapeutic uses. One well-known example is the case of the drug sildenafil citrate, which was repositioned for the treatment of erectile dysfunction while being studied for sildenafil citrate's primary indication of angina. Compared to traditional methods of drug development, drug repositioning advantageously provides reduced development time and decreased costs, as significant pharmacokinetic, toxicology, and safety data will have already been accumulated for existing drugs, reducing the risk of attrition during clinical trials.

Drug side-effects can be attributed to a number of molecular interactions, including on- or off-target binding, drug-drug interactions, dose-dependent pharmacokinetic, metabolic activities, downstream pathway perturbations, aggregation effects, and irreversible target binding. The side-effects caused by a drug can provide insight into the physiological changes that a drug causes—changes which can be difficult to predict using pre-clinical or animal models.

By determining the profiles of side effects that are caused by different drugs, it is possible to predict (and identify) chemically dissimilar drugs that share target proteins, based on the similarity of their side-effect profiles. Because drugs that have a significant number of side effects in common may share a common mechanism of action, the side-effect profile of a particular drug X can effectively be used to predict a phenotypic biomarker for the particular disease that drug X is designed to treat (for example, obesity). Thus, if drug Y (used to treat, for example, diabetes) also causes a distinct profile of side-effects that is highly correlated with the side-effect profile of drug X, drug Y should be evaluated for repositioning for the treatment of obesity.

As shown in FIG. 7, and as discussed above, the method begins with the step 710 of receiving textual messages from social media platform feeds, and then uses cascaded filters at step 720 to discard those posts which are not likely to be of interest to the system. The filtered messages are then classified at step 730, and classified posts that discuss drug-related side effects are stored at step 740. By comparing the side effects that have been caused by the various drugs discussed within the classified posts, the system can then calculate a correlation matrix using a global statistical model at step 750. The correlation matrix contains a correlation value for each pair of drugs discussed within the classified messages, indicating the degree of similarity between the side effects caused by each of those pair of drugs. A user may use these values to predict candidates for repositioning by selecting pairs of drugs having the highest correlation values. Additionally, at step 760, the system can generate a graphical model of a side-effect network, illustrating the varying correlations between drugs' side-effect profiles.

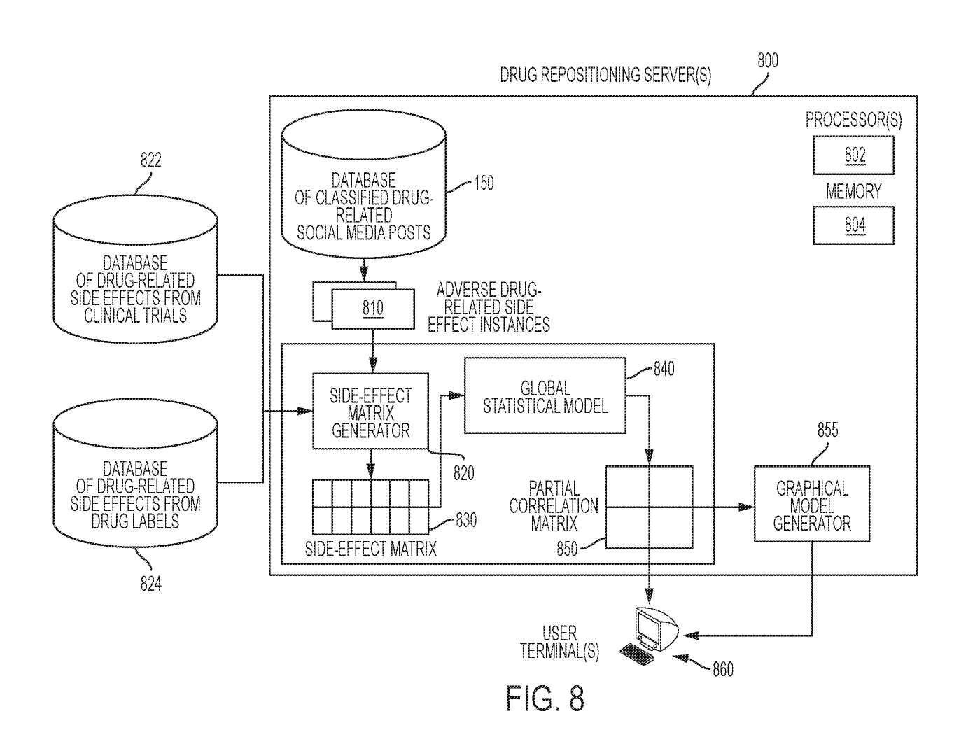

FIG. 8 is a diagram of an exemplary system for predicting candidates for drug repositioning using social media posts discussing drug-related side effects, and illustrates a system for using classified social media data to predict potential candidates for drug repositioning. The system operates on one or more drug repositioning server(s) 800, the drug repositioning servers 800 having one or more processors 802 and one or more memory units 804. The drug repositioning server 800 utilizes data from a database containing classified drug-related social media posts 150 (also depicted in FIG. 1) as discussed above. The database 150 provides a set of classified posts 810 discussing drug-related side effects (including both adverse and benign side effects) to system 800.

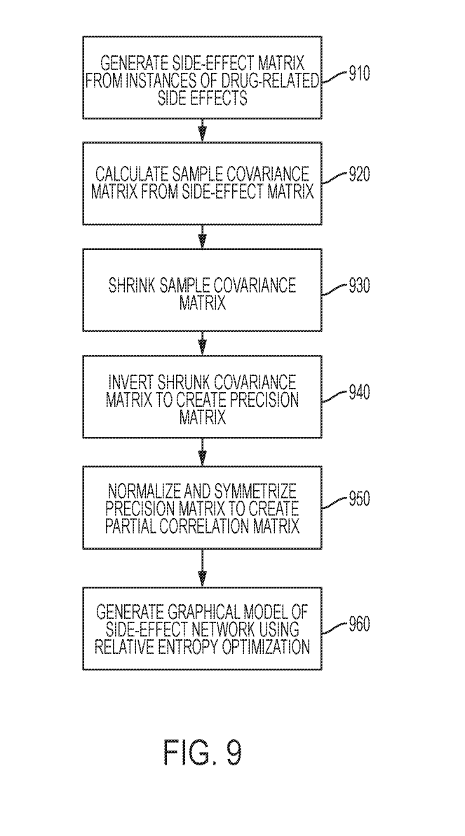

These classified posts 810 are input into side-effect matrix generator 820, which uses the drug and side effect data contained within posts 810 to generate a side-effect profile matrix 830, as depicted by step 910 in FIG. 9. Each column in the side-effect profile matrix 830 represents a unique drug, and each row in the side-effect profile matrix 830 represents a unique side effect. For example, if the set of classified posts 810 contains data on 620 unique drugs and 2196 unique side-effects, the side-effect matrix generator 820 will generate a 2196 column by 620 column matrix 830, with each cell in the matrix 830 containing a binary variable X. For each cell of matrix 830, if the drug represented by that column has been reported to cause the side effect represented by that row, X is set to 1. If the drug represented by that column has not been reported to cause the side effect represented by that row, X is set to 0.

In addition to posts 810 from database 150, the side-effect matrix generator 820 may also receive drug & side effect data from other sources. Such sources may include a database 822 containing drug-related side effect data recorded in clinical trials—for example, the Thomson Reuters CORTELLIS™ Clinical Trials Intelligence platform; and/or a database 824 containing drug-related side effect data from drug labels—for example, the SIDER database or the Thomson Reuters World Drug Index. These additional sources 822 and 824 can both provide additional side-effect data, as well as help identify false positive relationships between drugs and side effects that have been reported in posts 810.

Side-effect profile matrix 830 is then input into global statistical model 840, which calculates a sample covariance matrix S from the side-effect profile matrix 830, as shown in step 920 in FIG. 9. Each element S_{i,j} of the sample covariance matrix S represents the covariance of a first drug i with a second drug j. The sample covariance matrix S is calculated using the following formula:

$${S}_{i , j} = \frac{1}{n - 1} {\sum }_{k = 1}^{n} ( {x}_{ki} - {\overline{x}}_{i} ) ( {x}_{kj} - {\overline{x}}_{j} ) = \frac{1}{n - 1} {\sum }_{k = 1}^{n} {x}_{ki} {x}_{kj} - {\overline{x}}_{i} {\overline{x}}_{j}$$

In the above formula,

$${\overline{x}}_{i} = \frac{1}{n} {\sum }_{k = 1}^{n} {x}_{ki}$$

and xₖᵢ is the K-th side effect reported for drug Xᵢ. It can be shown that the average product of two binary variables (such as the binary variables contained within side-effect profile matrix 830) is equal to their observed joint probabilities such that:

$$\frac{1}{n - 1} {\sum }_{k = 1}^{n} {x}_{ki} {x}_{kj} = P ( {X}_{j} = 1 \vert {X}_{i} = 1 )$$

In the above equation, P(Xⱼ=1|Xᵢ=1) refers to the conditional probability that variable Xⱼ=one given that Xᵢ=one (that is to say, the probability that both drug j causes a side effect given that drug i causes that same side effect). Similarly, the product of the means of two binary variables (such as the binary variables contained within side-effect profile matrix 830) is equal to the expected probability that both variables are equal to one, under the assumption of statistical independence:

x̅ᵢ x̅ⱼ=P(Xᵢ=1)P(Xⱼ=1)

As a result, the covariance of two binary variables (such as the binary variables contained within side-effect profile matrix 830) is equal to the difference between the observed joint probability and the expected joint probability: S_{i,j}=P(Xⱼ=1|Xᵢ=1)−P(Xᵢ=1)P(Xⱼ=1)

The ultimate objective of global statistical model 840 is to invert sample covariance matrix S, producing a precision or concentration matrix θ which can be used to calculate the correlation between pairs of drugs. For the sample covariance matrix S to be easily invertible, it should have two desirable characteristics: 1) that it is positive definite (all eigenvalues of the matrix be distinct from zero); and 2) that it is well-conditioned (the ratio of its maximum and minimum singular value should not be too large). To promote these characteristics, and to speed up convergence of the inversion, the global statistical model 840 conditions the sample covariance matrix S by shrinking towards an improved covariance estimator T, as depicted in step 930 of FIG. 9.

Shrinking the sample covariance matrix S pulls the most extreme coefficients of matrix S towards more central values, thereby systematically reducing estimation error, by using a linear shrinkage approach to combine the estimator and sample matrix in a weighted average to create shrunk matrix S′:

S′=αT+(1−α)S

In the above equation, αε{0,1} denotes the analytically determined shrinkage intensity.

The shrunk matrix S′ is then inverted, as shown in step 940 of FIG. 9, resulting in precision or concentration matrix θ. Using inverted precision matrix θ, we can then obtain the matrix 850 of partial correlation coefficients ρ for all pairs of variables (the correlation between each possible pair of drugs), by using the following equation, as shown in step 950 of FIG. 9:

$${ρ}_{i , j} = \frac{{θ}_{i , j}}{\sqrt{{θ}_{i , i} {θ}_{i , j}}}$$

The matrix 850p will have a number of rows and columns equal to the number of drugs in side-effect profile matrix 830. The partial correlation between two drugs (X and Y) given a third drug Z can be defined as the correlation between the residuals Rₓ and R_{y} after performing least-squares regression of X with Y and Z, respectively. This value, denoted as p_{x,y|z} provides a measure of the correlation between drugs X and Y when conditioned on the third drug Z, with a value of zero implying conditional independence between drugs X and Y if the input data distribution is multivariable Gaussian. The partial correlation matrix 850 ρ gives the correlation between all pairs of drugs conditioning on all other drugs. Off-diagonal elements in matrix 850 ρ that are significantly different from zero will therefore be indicators of pairs of drugs that show unique covariance between their side-effect profiles, after taking into account (such as by removing) the variance of side-effect profiles amongst all the other drugs.

A desired output from the global statistical model 840 is a sparse partial correlation matrix 850 that contains many zero elements, as it is known that relatively few drug pairs will share a common mechanism of action. Therefore, removing any spurious correlations between pairs of drugs (and replacing them with zero elements) is desirable and results in a more parsimonious relationship model, with the remaining non-zero elements in matrix 850 more likely to reflect correct positive correlations between pairs of drugs. However, elements of matrix 850 are unlikely to be zero unless many elements in the sample covariance matrix S are also zero. A statistical method known as the “graphical lasso” is therefore used to induce zero partial correlations in matrix 850, by penalizing the maximum likelihood estimate of the inverted precision matrix θ using a l₁-norm penalty function to produce an estimate of a sparse inverted matrix. The estimate can be found by maximizing the following log-likelihood:

log detθ−tr(S′θ)−λ|θ|₁

The first term in the above equation is the Gaussian log-likelihood of the data, tr denotes the trace operator, and ∥θ∥₁ is the l₁-norm—the sum of the absolute values of the elements of the inverted precision matrix θ, weighted by the non-negative tuning parameter A. The specific use of the lᵢ-norm penalty has the desirable effect of setting elements in θ to zero, while the parameter λ effectively controls the sparsity of the solution. The value of tuning parameter λ may range from approximately 10⁻⁷ to 10⁻¹². In certain embodiments, a value of 10⁻⁹ is used for tuning parameter λ.

The graphical lasso method described above produces an approximation of matrix θ that is not symmetric, so it must be updated as follows:

$$θ ← \frac{( θ + {θ}^{T} )}{2}$$

After updating the inverted precision matrix θ, the partial correlation matrix 850 ρ can then be calculated in step 950, using the following equation as described above:

$${ρ}_{i , j} = \frac{{θ}_{i , j}}{\sqrt{{θ}_{i , i} {θ}_{i , j}}}$$

The resulting partial correlation matrix 850 will therefore contain correlation values for each possible pair of drugs, indicating the correlation between the side effect profiles of each drug of the pair of drugs, and will have a number of rows and columns equal to the number of drugs for which correlations have been calculated. As described above, if the matrix 850 calculates correlations between the side effect profiles of 620 drugs, for example, matrix 850 will have 620 rows and 620 columns, with each row representing a unique drug and corresponding to a column that also represents that unique drug.

Matrix 850 can be output by server 800 to user terminal 860, allowing a user at terminal 860 to view the correlation data contained within matrix 850. User terminal 860 may request, for example, the top 5, 10, 25, or 50 candidates for repositioning for drug X—which correspond to the drugs represented by the columns intersecting the cells with the top 5, 10, 25, or 50 values in row X of matrix 850. For example, if the highest partial correlation value in row X (corresponding to drug X) of matrix 850 is located in the cell where row X intersects column Y (corresponding to drug Y), that indicates that drug X may be a candidate for repositioning to treat the medical condition targeted by drug Y (and, vice versa, that drug Y may be a candidate for repositioning to treat the medical condition targeted by drug X).

In addition to determining options for repositioning of an individual drug, server 800 may also output repositioning candidates for a particular condition to user terminal 860. For example, if ten of the drugs in matrix 850 were associated with diabetes, server 800 could output the 5/10/25/50 highest correlation coefficients found in matrix 850's ten rows representing those ten diabetes drugs. The drugs that correspond to the columns in which those highest correlation coefficients are found will be the top potential candidates for repositioning to treat diabetes.

Server 800 also features a graphical model generator 855, which can be used to generate a graphical representation of matrix 850 to be displayed on a display screen of user terminal 860. In certain embodiments, the graphical model generator 855 generates a graphical depiction of a side-effect network that represents all drugs and correlations between drugs contained in matrix 850, as shown in step 960 of FIG. 9.

The side-effect network contains nodes, representing drugs, and edges between nodes, representing correlations between the side-effect profiles of those drugs. In certain embodiments, the display of the side-effect network can be generated using scalable vector graphics, and the layout of the nodes and correlations in the display can be determined using a relative entropy optimization-based method.

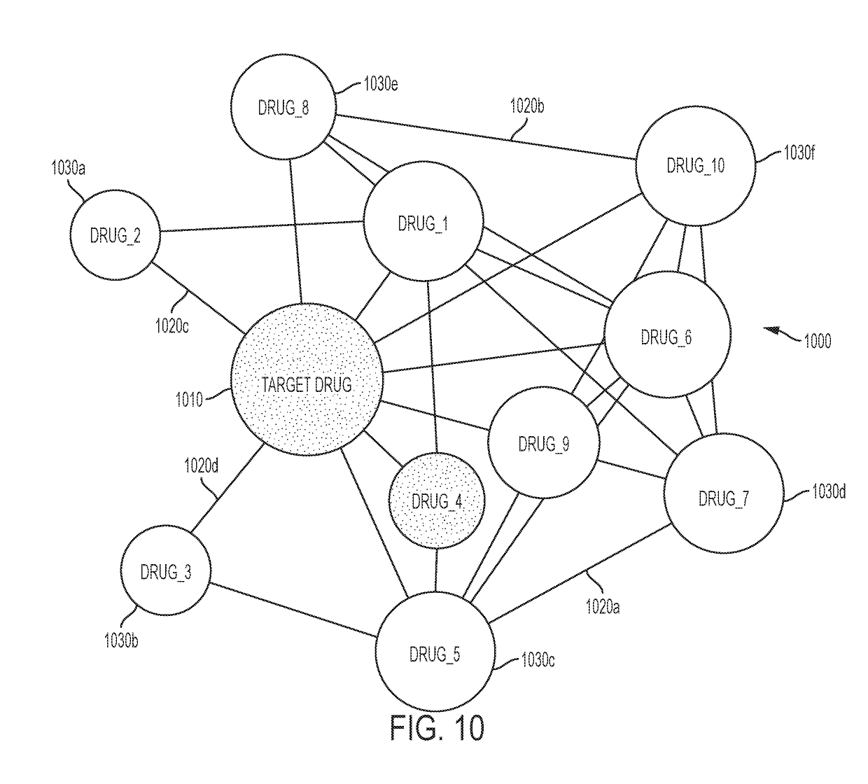

In certain embodiments, the graphical model generator 855 is configured to allow a user of terminal 860 to select an individual node (representing a drug) in the side-effect network, and to generate a view, such as exemplary display 1000 of FIG. 10, generated by the graphical model generator of the system of FIG. 8. Display 1000 is centered around target node 1010, and displays edges 1020 representing correlations between target node 1010 and candidate nodes 1030. In certain embodiments, the graphical model generator 855 can directly generate display 1000 for a target drug from matrix 850 without first displaying a graphical model of a side-effect network representing and displaying all the drugs and correlations within matrix 850.

In display 1000, nodes 1010 and 1030 have been arranged using a force-directed layout approach, so that the nodes 1010 and 1030 are as equidistantly positioned as possible, and so there are as few crossings between edges 1020 as possible. The display 1000 not only displays edges 1020 between target node 1010 and candidate nodes 1030 (e.g., edges 1020c and 1020d), but also edges 1020 between candidate nodes 1030 themselves (e.g., edges 1020a and 1020b)

Nodes 1010 and 1030 can be sized based on the number of correlations 1020 displayed for a certain node 1010 or 1030—thus, node 1010, connected to nine edges 1020, has a larger diameter than node 1030a or 1030b, each of which is only connected to two edges. The thickness of an edge 1020 can be proportional to the value of the correlation coefficient it represents. For example, the higher thickness of edge 1020a as compared to edge 1020b represents a higher correlation coefficient between the drugs represented by nodes 1030c and 1030d as compared to the lower correlation coefficient between the drugs represented by 1030e and 1030f. That is, a thicker edge 1020a represents a higher probability that each drug 1030c and 1030d in the pair connected by that edge 1020a is a candidate for repositioning to treat the condition targeted by its counterpart.

The structures shown and discussed in embodiments of the invention are exemplary only and the functions performed by these structures may be performed by any number of structures. For example, certain functions may be performed by a single physical unit, or may be allocated across any number of different physical units. All such possible variations are within the scope and spirit of embodiments of the invention and the appended claims.

Embodiments of the present invention have been described for the purpose of illustration. Persons skilled in the art will recognize from this description that the described embodiments are not limiting, and may be practiced with modifications and alterations limited only by the spirit and scope of the appended claims which are intended to cover such modifications and alterations, so as to afford broad protection to the various embodiments of invention and their equivalents.

What is claimed is:

1. A pharmacovigilance system for filtering and classifying social media textual messages that include pharmacological content, comprising:
a plurality of cascading filters configured to receive a plurality of textual messages that contain at least one keyword of a plurality of keywords related to pharmaceuticals, wherein the plurality of textual messages are input into a first cascading filter, each of the plurality of cascading filters evaluates whether textual messages input into that filter satisfy a criterion of that filter and each of the plurality of cascading filters outputs a subset of textual messages that satisfy the criterion of that filter, so that a last cascading filter outputs a final subset of the plurality of textual messages;
a feature extractor that receives the final subset of textual messages, extracts a vector of features from each textual message of the final subset, and outputs the final subset of textual messages and an associated vector of features for each message of the final subset;
a classifier comprising a machine learning model that receives the vector of features, determines whether the textual message associated with each vector of features belongs to a particular class associated with the machine learning model, and provides an output of one or more textual messages that belong to that particular class to an indexed database of classified textual messages, wherein the particular class comprises messages related to adverse drug-related side effects;
an indexed database of classified textual messages that stores the classified textual messages, a particular class associated with each of the classified textual messages, and metadata associated with each of the classified textual messages; and
an application programming interface configured to access the indexed database and to provide stored classified textual messages and metadata associated with those classified textual messages to at least one customer application configured to allow customers to view the classified textual messages and metadata.

2. The system of claim 1, wherein the plurality of textual messages are user posts to at least one social media platform, and the plurality of textual messages are provided by the at least one social media platform.

3. The system of claim 1, wherein the plurality of textual messages comprises textual messages that contain at least one word that matches at least one morphological structure.

4. The system of claim 1, wherein the plurality of cascading filters comprises a filter that outputs original textual messages and discards textual messages that are copies of the original textual messages.

5. The system of claim 1, wherein the plurality of cascading filters comprises a filter that outputs textual messages written in a first language, and discards textual messages that are not written in that first language.

6. The system of claim 1, wherein the plurality of cascading filters comprises a filter that outputs textual messages written in a language that is a member of a group of languages, and discards textual messages that are written in a language that is not a member of that group of languages.

7. The system of claim 1, wherein the plurality of cascading filters comprises a filter that outputs textual messages that do not contain a hyperlink, and discards textual messages that contain hyperlinks.

8. The system of claim 1, wherein the plurality of cascading filters comprises a filter that outputs textual messages that contain at least one keyword from a list of keywords, and discards textual messages that do not contain any of the keywords in the list of keywords.

9. The system of claim 1, wherein the plurality of cascading filters comprises a filter that outputs textual messages that contain at least one word that matches at least one morphological structure, and discards textual messages that do not contain any words that match the at least one morphological structure.

10. The system of claim 1, comprising a database that stores the final subset of textual messages output by the plurality of cascading filters.

11. The system of claim 1, wherein the vector of features extracted from the textual message comprises one or more surface features comprising: a) the number of characters in the textual message divided by a maximum length limit of the textual message; b) the number of social media usernames in the textual message; c) the maximum number of times an alphanumeric character is repeated within a word in the textual message; d) whether the textual message contains at least one numerical character; e) whether the textual message contains at least one upper-case word; f) whether the textual message contains at least one title-case word; and g) if the textual message contains at least one word with mixed capitalization.

12. The system of claim 1, wherein the vector of features extracted from the textual message comprises one or more part-of-speech tag features comprising: a) whether the textual message contains verbs in both past and present tense; b) whether the textual message contains a wh-determiner, a wh-pronoun, a possessive wh-pronoun, or a wh-adverb; c) whether the textual message contains a comparative or superlative adverb; and d) a concatenation of all verb part-of-speech (POS) tags in the textual message in the alphabetical order of those verb POS tags.

13. The system of claim 1, wherein the vector of features extracted from the textual message comprises one or more gazetteer features comprising: a) whether the textual message contains at least one word or phrase listed in a gazetteer containing a list of words and phrases; b) the number of words and/or phrases in the textual message matching words or phrases in the gazetteer; and c) the percentage of the alphanumeric characters in the textual message that are contained in the words and/or phrases in the textual message matching words or phrases in the gazetteer.

14. The system of claim 13, wherein the gazetteer containing a list of words and phrases comprises: a) a user vocabulary gazetteer comprising words and phrases indicating abuse, humor, fiction, intake, efficacy, and patient feedback about a drug; b) a company gazetteer comprising words and phrases related to commercial spam, commercial pharmaceutical companies, financial and share price information, company news, and company designators; and c) a medical vocabulary gazetteer comprising words and phrases related to human body parts, adverse effect symptoms, side effect symptoms, adverse events, casuality indicators, clinical trials, medical professional roles, side effect triggers, and drugs.

15. The system of claim 1, wherein the vector of features extracted from the textual message comprises one or more sentiment features comprising: a) the number of words in the textual message having a negative sentiment value; b) the sum of the negative sentiment values of the words having a negative sentiment value; c) the average negative sentiment value of the words having a negative sentiment value; d) the number of words in the textual message having a positive sentiment value; e) the sum of the positive sentiment values of the words having a positive sentiment value; and f) the average positive sentiment value of the words having a positive sentiment value.

16. The system of claim 1, wherein the machine learning model is a support vector machine.

17. The system of claim 16, wherein the machine learning model has been trained using a set of training data, the set of training data comprises a plurality of sample textual messages, and the sample textual messages are each manually annotated as positive or negative examples of the particular class associated with the machine learning model.

18. The system of claim 1, wherein the classifier comprises a plurality of machine learning models, and each of the plurality of machine learning models is associated with and optimized for a particular class of textual messages.

19. The system of claim 18, wherein the plurality of machine learning models are cascading classifiers.

20. The system of claim 18, wherein the plurality of machine learning models are parallel-voting classifiers.

21. The system of claim 1, wherein the classifier comprises a plurality of machine learning models, and each of the plurality of machine learning models is optimized for a particular language.

22. The system of claim 1, wherein the indexed database comprises classified textual messages of a single particular class.

23. The system of claim 1, wherein the indexed database comprises classified textual messages of a plurality of classes.

24. The system of claim 1, wherein the metadata associated with the classified textual messages comprises: a) a time at which the textual message was posted on a social media platform; b) the geographical location from which the textual message was posted; c) at least one class to which the textual message belongs; and d) a gender of a composer of the textual message.

25. The system of claim 1, wherein the at least one customer application is configured to generate a computerized display that allows a customer to indicate whether the customer believes a particular classified textual message was properly classified.

26. The system of claim 1, wherein the at least one customer application is configured to generate one or more computerized displays comprising: a) one or more classified textual messages from the classified textual messages stored in the indexed database; b) a timeline displaying the time at which a plurality of classified textual messages were posted on at least one social media platform; c) a geographical map displaying the geographical locations from which a plurality of classified textual messages were posted; and d) a chart or graph indicating the number of classified textual messages that are associated with a specific class of a plurality of classes, wherein each of those classified textual messages contains a particular keyword or phrase.

27. A pharmacovigilance method for filtering and classifying social media textual messages that include pharmacological content, comprising:
training at least one machine learning model to identify textual messages that are positive examples of a particular class associated with the at least one machine learning model, wherein the particular class comprises messages related to adverse drug-related side effects;
receiving a plurality of textual messages containing at least one keyword from at least one source of textual messages;
inputting the received plurality of textual messages into a plurality of cascading filters, filtering out textual messages that do not satisfy a respective criterion of each of those plurality of cascading filters, and outputting a final subset of textual messages that satisfy the criteria of all the cascading filters;
extracting a vector of features from each of the final subset of textual messages, and associating each vector of features with the respective textual message from which it was extracted;
inputting the vectors of features into the at least one trained machine learning model, and classifying the textual message associated with each vector of features as a positive or negative example of a message related to adverse drug-related side effects;
indexing and storing one or more textual messages classified as a positive example and metadata associated with those one or more textual messages in an indexed database; and
providing the one or more textual messages classified as a positive example of a message related to adverse drug-related side effects and the metadata associated with the one or more textual messages to a customer application configured to allow customers to view classified textual messages and metadata.

28. The method of claim 27, wherein training the at least one machine learning model comprises training the at least one machine learning model with a training set comprising a plurality of sample textual messages that have been manually annotated as negative or positive examples of the particular class associated with the at least one machine learning model.

29. The method of claim 27, wherein training the at least one machine learning model further comprises surrogate training of the at least one machine learning model using automatically selected sample textual messages, and wherein the automatically selected sample textual messages are automatically selected as likely positive examples of the particular class associated with the at least one machine learning model.

30. The method of claim 27, wherein training the at least one machine learning model comprises receiving feedback on whether the indexed and stored classified textual messages were properly classified.

31. The method of claim 27, wherein the at least one machine learning model comprises a support vector machine, and wherein training the at least one machine learning model comprises grid search optimization of the at least one support vector machine.

32. The method of claim 27, wherein receiving the plurality of textual messages from at least one source of textual messages comprises receiving a stream of textual messages from at least one social media platform.

33. The method of claim 27, wherein receiving the plurality of textual messages from at least one source of textual messages comprises receiving streams of textual messages from a plurality of different social media platforms.

34. The method of claim 27, wherein the received plurality of textual messages comprise textual messages that contain at least one keyword from a list of keywords.

35. The method of claim 27, wherein filtering out textual messages that do not satisfy a respective criterion of each of those plurality of cascading filters comprises at least one of: a) filtering out all textual messages that are copies of original textual messages; b) filtering out all textual messages that are not written in one or more particular languages; c) filtering out all textual messages that do not contain a hyperlink; and d) filtering out all textual messages that do not contain at least one word or phrase from a list of key words and phrases.

36. The method of claim 27, wherein extracting a vector of features comprises tokenizing and normalizing the textual message.

37. The method of claim 27, wherein extracting a vector of features comprises extracting one or more N-gram features comprising: a) unigrams containing only alphanumeric characters; and b) bigrams containing only alphanumeric characters.

38. The method of claim 27, wherein inputting the vectors of features into the at least one machine learning model comprises inputting the vectors of features into a single machine learning model.

39. The method of claim 27, wherein inputting the vectors of features into the at least one machine learning model comprises inputting the vectors of features into a plurality of machine learning models, and wherein the plurality of machine learning models comprise cascading machine learning models or parallel-voting machine learning models.

40. The method of claim 27, wherein the metadata associated with the one or more classified textual messages comprises: a) a time at which the textual message was posted on a social media platform; b) the geographical location from which the textual message was posted; c) at least one class to which the textual message belongs; and d) a gender of a composer of the textual message.

41. A system for determining candidates for drug repositioning, comprising:
a set of cascading filters configured to receive a plurality of social media posts and to output a subset of filtered social media posts from the set of cascading filters;
a classifier configured to receive the subset of filtered social media posts from the set of cascading filters and to output a subset of classified social media posts related to drug-related side effects from the classifier;
a database configured to receive the subset of classified social media posts from the classifier and to store the subset of classified social media posts;
a side-effect profile matrix generator configured to retrieve the subset of classified social media posts from the database, and to generate a side-effect profile matrix representing the side-effects associated with a plurality of drugs from the subset of classified social media posts; and
a global statistical model configured to receive the side-effect profile matrix from the side-effect matrix generator and to output a correlation matrix comprising correlations between pairs of drugs from the plurality of drugs.

42. The system of claim 41, further comprising a graphical model generator configured to generate a graphical display of a side-effect network from the correlation matrix.

43. The system of claim 42, wherein the graphical display comprises a plurality of nodes and a plurality of edges between pairs of nodes, each node represents a drug, and each edge between a pair of nodes represents the correlation between a first side-effect profile of a first drug and a second side-effect profile of a second drug.

44. The system of claim 43, wherein a thickness of an edge between a pair of nodes represents the strength of the correlation between the first side-effect profile and the second side-effect profile.

45. A method for computing candidates for drug repositioning, comprising:
receiving a plurality of drug-related side effect descriptions, each description comprising: a) a drug; and b) a side effect resulting from the drug;
generating a side-effect matrix from the drug-related side effect descriptions;
generating a sample covariance matrix from the side-effect matrix;
shrinking the sample covariance matrix to create a shrunk covariance matrix;
inverting the shrunk covariance matrix to create a precision matrix;
normalizing and symmetrizing the precision matrix to create a partial correlation matrix; and
ranking drug repositioning candidates from the partial correlation matrix.

46. The method of claim 45, wherein each row in the side-effect matrix represents a side effect, each column in the side-effect matrix represents a drug, and each cell in the side-effect matrix represents whether a particular side effect has been reported for a particular drug.

47. The method of claim 45, wherein each row in the partial correlation matrix represents a drug, each column in the partial correlation matrix represents a drug, and each cell in the partial correlation matrix represents the correlation calculated between a first drug and a second drug.

48. The method of claim 45, wherein shrinking the sample covariance matrix comprises applying a distribution-free, diagonal, unequal variance model.

49. The method of claim 45, wherein inverting the shrunk covariance matrix comprises using a graphical lasso.

50. The method of claim 45, further comprising generating a drug side-effect network from the partial correlation matrix using a relative entropy optimization-based method.
